(19) United States
(12) Patent Application Publication
(10) Pub. No.: US 2011/0223191 A1
(43) Pub. Date: Sep. 15, 2011

(54) ANTI-CANCER NUTRACEUTICAL COMPOSITION

(75) Inventors: Suhaila Mohamed, Serdang (MY); Farideh Namvar, Serdang (MY); Kheen Kuan Chan, Serdang (MY)

(73) Assignee: UNIVERSITI PUTRA MALAYSIA, SELANGOR (MY)

(21) Appl. No.: 13/128,537

(22) PCT Filed: Dec. 23, 2009
(86) PCT No.: PCT/MY2009/000213

§ 371 Date: May 10, 2011

(30) Foreign Application Priority Data
Dec. 23, 2008 (MY) PI 20085248

(51) Int. Cl. A61K 36/02; A61K 36/889; A61K 36/67; A61K 36/81; A61K 36/04; A61K 36/05; A61K 36/03; A61P 35/00

(52) U.S. Cl. 424/195.17; 424/727; 424/734; 424/725

(57) ABSTRACT

A comestible composition with anti-cancer property comprises alcohol soluble extracts from plant parts of Piper species, palm species, tropical seaweeds, and/or Solanum species.

### FIELD OF INVENTION

The present invention relates to a nutraceutical composition with significant anti-cancer property. In more specific, the disclosed composition contains extract from plant parts of the Piper species, tropical seaweeds, palm and/or Solanum species.

### BACKGROUND OF THE INVENTION

Unrestrained cell proliferation is the characteristic of cancer or tumor cells with damaged genes that directly control their cell cycles. Changes in cell survival contribute to a number of human diseases, including cancer, viral infections, autoimmune diseases, neurodegenerative disorders, and AIDS (acquired immunodeficiency syndrome). Natural physiological cell death occurs primarily through apoptosis. Apoptosis is the natural process of removing cells in normal or pathologic tissues. Rapid condensation and budding of the cell occur, with the formation of membrane-enclosed apoptotic bodies containing well-preserved organelles, which are phagocytosed and digested by adjacent local cells, without any inflammation. Apoptosis takes place spontaneously in malignant tumors, often significantly retarding their growth, and are increased in tumors responding to irradiation, cytotoxic chemotherapy, heating and hormone ablation. Apoptosis can be regulated by certain proto-oncogenes and the p53 tumor suppressor gene. C-myc expressions are involved in the initiation of apoptosis in some situations, and bcl-2 is a new type of proto-oncogene that inhibits apoptosis, rather than stimulating mitosis. Antibodies against a cell-surface protein designated APO-1 or Fas can enhance apoptosis in some human lymphoid cell lines which may have therapeutic uses.

Apart from that, it was found nutraceuticals derived from certain parts of plants also capable of causing apoptosis to cancer cells while these nutraceuticals are admisnistered to the subject at an effective amount. Thus retarding the progression or even leading to the regression of cancer.

Different metabolites from various plants have been proven to possess anti-cancer properties. Subsequently, different anti-cancer products containing these metabolites as the active ingredients have been developed. For example, Kimura et. al. filed a Japanese patent application no. 2006306897 which are directed to provide a new anti-gastric cancer agent that suppresses gastric cancer cell proliferation which employs the fucoidan originating from Mozuku seaweed as the effective component. The fucoidan originating from hot water extract of Mozuku seaweeds, for example, Ito mozudu (Nemacystus decipieus), Okinawa mozuku (Cladosiphon okamuranus) or Futo mozuku (Tinocladia crassa) or the like or a purified product thereof obtained by treating the extract with a quaternary ammonium salt.

Another United States patent application no. 2003015712 filed by Iwasaki, Teruaki to disclose a nutritious supplemental composition for suppression against onset of large intestinal cancer and manufacturing method thereof. The invention provided a composition containing well-balanced nutrition, having an effect to suppress the mutagenesis substances. The disclosed invention claims to have no adverse reaction even if the composition is continued to be taken as nutritious supplemental substance and capable of promoting healthy state. The composition preferably contains dietary fibre in a range of 15 wt % to 30 wt % in respect to a total amount of composition. The dietary fibre contains in the form of dried koji fine powder including dead fungi of Aspergillus.

Another U.S. Pat. No. 4,708,962 provides an antiviral and antitumor cyclohexadienone compositions. Besides, a method for inhibiting, remitting or controlling the growth of tumors or tumor cells utilizing the disclosed compositions is provided as well. More particularly, the antitumor compositions comprise, as active ingredient, an antitumor effective amount of halogenated chamigrenes extracted and derived from red alga and sea hares which diet upon red alga.

In United State patent application no. 2006228432, an alpha-glucosidase inhibitors is disclosed and a method to obtain the claimed subject. The claimed alpha-glucosidase inhibitors is actually an extract derived from the plant source of Piper longum and acquired through a polar solvent. Owing to its property in inhibiting alpha-glucosidase activity, the extract is believed in preventing progression of cancer cells.

European patent application 1508334 claims a water soluble extract from plant of solanum genus and a pharmaceutical product containing the extract as an active ingredient in inhibiting growth of tumor/cancer cells, particularly liver cancer cells, lung cancer cells and breast cancer cells.

Another Japan patent publication no. 2004091472 discloses an apoptosis inducer contained in potato anthocycnin comprises the anthocyanin pigment-containing extract. The extract is acquired from agricultural species such as Solanum tuberosum, Andigena L and S. phureja.

### SUMMARY OF THE INVENTION

The present invention aims to provide a neutraceutical composition which is effective against cancer cells. In more specific, the neutraceutical composition is capable of inducing apoptosis in cancer cell line without affecting growth of normal cells.

Further object of the present invention is to provide a method to acquire the above mentioned composition through an extraction method.

At least one of the preceding objects is met, in whole or in part, by the present invention, in which one of the embodiment of the present invention a composition with anti-cancer property comprising alcohol soluble extract from vegetative parts of Piper species, palm species, tropical edible seaweed, and/or Solanum spp. In the most preferred embodiment, the Piper species is Piper betel to be used in the present invention.

In further embodiment, the disclosed composition may contain also alcohol soluble extract from palm leaf, Solanum species and/or tropical seaweeds. Likewise, the Solanum species is any one or combination of (but not limited to) S. mammosum, S. verbascifolium, S. tuberosum, S. trilobatum, S. torvum, S. seaforthianum, S. sarmentosum, S. nigrum, S. melongena, S. maroniense, S. microcarpon, S. jasminoides, S. indicum, S. ferox, S. coagulans, S. blumei, S. aculeatissimum, while the tropical seaweeds are any one or combination of Eucheuma cottonii, Caulerpa lentillifera, and Sargassum polycystum.

In one of the aspect, the Palm species is Elaeis guineensis, Elaeis oleifera, Phoenix dactylifera and Cocos nucifera. Moreover, it is the leaves of these species that are used for extracting the useful compounds.

Another major aspect of the present invention includes a method for obtaining alcohol soluble extract with anti-cancer property from plant parts of Piper species, palm leaves, Solanum species and/or tropical seaweeds comprising the steps of pre-treating plant parts of the Piper species, palm leaves, plant parts of the Solanum species and/or tropical seaweeds; subjecting the pre-treated plant parts for extraction using a first polar extraction solvent comprising an alcohol, with or without a co-solvent; and concentrating the extraction solvent/s to form a powdery composition or paste after removing the pre-treated plant parts. In respect to the preferred embodiment, the Piper species is Piper betel, palm leaf, tropical seaweeds and the Solanum species is any one or combination of S. mammosum, S. verbascifolium, S. tuberosum, S. trilobatum, S. torvum, S. seaforthianum, S. sarmentosum, S. nigrum, S. melongena, S. maroniense, S. microcarpon, S. jasminoides, S. indicum, S. ferox, S. coagulans, S. blumei, S. aculeatissimum.

To effectively extract metabolites from the plant parts, the ratio of weight of the pre-treated plant parts to the first polar extraction solvent is preferably in a range of 1-3: 6-10 (w/v).

### BRIEF DESCRIPTION OF THE DRAWINGS

FIG. 1 is a graph showing the Percentages of apoptotic breast cancer MCF-7 cells in the presence of Sargassum polycystum alcoholic extract SPME (0-200 μg/ml) compared to normal cells;

FIG. 2 is a graph showing the Reduction of viability of five human cancer cells after 48 h incubation, measured by MTT test (mean±S.D.; n=3) by each seaweeds alcoholic dry extracts (concentration 12.5 to 200 μg/ml));

FIG. 3 are microscope photographs showing morphological changes of breast cancer MCF-7 cells after treatment by S. polycystum extract followed by Hoechst 33342 staining which (a) Fluorescence microscope photographs of control cells treated with 0.1% DMSO, (b) cells treated with 25 μg/ml S. polycystum extract after 24 h incubation;

FIG. 4 is a graph showing the viability of MDA 435 and MCF 7 breast cancer cells after incubating in various concentrations of extracts of Piper spp., Turmeric (Kunyit), and Solanum mammossum Terung Susu Kambing (TSK) for 48 hrs and 72 hours respectively (MTT Assay); and

FIG. 5 is a graph showing the viability (MTT assay) and Cell Death (LDH assay) of DU 145 Human Prostate cancer cells after incubating in various concentrations of extracts of Piper spp., for 24-72 hrs respectively (MTT Assay).

### DETAILED DESCRIPTION OF THE INVENTION

One skilled in the art will readily appreciate that the present invention is well adapted to carry out the objects and obtain the ends and advantages mentioned, as well as those inherent therein. The embodiment describes herein is not intended as limitations on the scope of the invention.

The term “pharmaceutically effective amount” used herein through out the specification refers to the amount of the active ingredient, the extract, to be administered orally to the subject to trigger the desired effect without or causing minimal toxic adverse effect against the subject. One skilled in the art should know that the effective amount can vary from one individual to another due to the external factors such as age, sex, diseased state, races, body weight, formulation of the extract, availability of other active ingredients in the formulation and so on.

With respect to the preferred embodiment of the present invention, a method for preparing extracts from plant parts of the tropical seaweeds, palm leaf, Solanum spp. fruits and Piper spp. is disclosed. The method basically comprises the steps of pre-treating plant parts from Tropical seaweeds, palmae family, Solanum spp. and Piper spp. extracting the pre-treated parts using a solvent, (with or without a co-solvent); removing the pre-treated parts from the used solvent; and concentrating the used solvent to acquire the herbal extract. The disclosed method is found to be effective in isolating the favored compounds, peptides or active metabolites which provides a anti-cancer enhancement effects or health promoting effect towards the injured system upon ingestion orally or topically of a subject. In particular, method for obtaining alcohol soluble extract with anti-cancer property from plant parts of Piper species, palm species, Solarium species and/or tropical seaweeds comprising the steps of pre-treating plant parts of the Piper species, palm species, Solanum species and/or tropical seaweeds; subjecting the pre-treated plant parts for extraction using alcohol (with or without the presence of a co-solvent); concentrating the extraction solvent to form a powder or paste.

In one of the aspect, the Palm species is Elaeis guineensis, Elaeis oleifera, Phoenix dactylifera and Cocos nucifera. Moreover, it is the leaves of these species that are used for extracting the useful compounds.

According to another preferred embodiment of the disclosed method, the plant parts are pre-treated before subjecting to extraction. The pre-treatment process may involve a washing step to clean any dirt or physical pollutants captured at the surface of the alcoholic extracts. Other pre-treatment step may include reducing the moisture content of the plant parts by any known means or approaches in the art to improve the extraction rate and yield. In the preferred embodiment, the plant parts are subjected to drying in an oven at 40-110° C. The drying temperature shall not be set too high as such practice may degrade the active metabolites and compounds contained within the alcoholic extracts of. The preferred temperature shall range from 40° C. to 70° C. Other pre-treatment steps that can be employed are preparing the dried plant parts into small fragments, or pulverization to paste or powdery form prior to the soaking step or extraction. The fragmentized portion or pulverized plant parts can greatly increase the available contact surface of the dried plant parts that are exposed to extraction solvent thus enhancing the rate as well as the yield of the extraction method.

Preferably, the pre-treated plant parts may be subjected to a plurality occasion of extraction using different types of extraction solvent to obtain the optimal yield. Though mere soaking the pre-treated plant parts into the extraction solvent shall be able to extract the active compounds due to the polarity attraction of the extraction solvent, the process may be accelerated by stirring the extraction mixture, or the use of heat and pressure, both of the extraction solvent and the pre-treated alcoholic extracts of, during the time the extraction is conducted. It is important to note that the extracted compounds from the alcoholic extracts of are mainly constituted of bio-phenols, proteins, pigments, minerals, polysaccharides, lipids, small peptides and other bioactive compounds. Therefore, the efficiency rate of the extraction may be sensitive towards pH changes in the extraction solvent. In the most preferred embodiment of the disclosed method, the pH of the extraction content shall fall within pH 3 to 9.5 for achieving optimal yield and extraction rate. Applying appropriate amount of heat energy to the extraction system is another feasible approach to enhance the extraction rate and yield. Relying upon the solvent utilized, the heating is most preferred within the range of 40° C. to 120° C., most preferably 40° C. to 90° C. Precaution should be taken into consideration that denaturalisation possibly occurs at high temperature of heating.

It is important to note that the extracts used in the disclosed method in the preferred embodiment derives from the plant species of but not limited to, Euchema cottonii, Appaphycus alvareziii, Caulerpa lentillifera, Sargassum polycystum, Piper species, Arecaceae family and Solanum species. The extracts obtained from the abovementioned plant species are suitable to be incorporated into edible or topical products, or as capsules, ointments, lotions and tablets.

The desired compounds to be extracted from the alcoholic extracts of are mainly constituted of, but not limited to, biophenols, proteins, lipids, saccharides, minerals and small peptides. Due to polarity of these compounds, the polar solvent such as alcohol is found to be effective in extracting these desired compounds from the plant matrix. To acquire optimal yield by using effective amount of extraction solvent, the ratio the pre-treated plant parts to the extraction solvent is preferably 1-3: 6-10 (w/v). More preferable, the plant parts of the Piper species and Arecaceae family are leaves.

Preferably, the pre-treated plant parts after subjecting to extraction can be separated by any known means and approaches in the art for disposal. Vacuum filtration is most preferred as such approach is commonly available. Similarly, vaporization of the used extraction solvent to concentrate compounds can be performed by different approaches. For example, drying the used extraction solvent using heat energy or vacuum drying. Concentrating the compounds by dissipating the used extraction solvent shall finally reach to the stage where the compounds are concentrated to a paste or powdery form. This paste or powdery form of compounds extract can then be utilized for various applications.

Attention is now drawn to another embodiment of the present invention which involves a comestible and topical composition with anti-cancer property comprising alcohol soluble extract derived the plant parts of Tropical seaweeds, Solanum spp. fruits and Piper spp. leaves and Palmae family using an appropriate extraction solvent. The comestibles mentioned herein can be any common daily consumed processed food such as bread, noodles, confections, chocolates, beverages, and the like. One skilled in the art shall appreciate the fact that the aforesaid extract can be incorporated into the processed comestibles, capsules, tablets or topical medicine during the course of processing. Therefore, any modification thereon shall not depart from the scope of the present invention.

As setting forth in the above description, the composition with anti-cancer property comprising alcohol soluble extract from leaves of Piper and Palm species. Apart from that the composition may further comprise alcohol soluble extract derived from the plants of tropical seaweeds and Solanum species. More preferably, the plant is any one or combination of the plant species of, but not limited to, Euchema cottonii, Appaphycus alvareziii, Caulerpa lentillifera, Sargassum polycystum, Elaeis guineensis, Elaeis oleifera, Phoenix dactylifera and Cocos nucifera, Piper spp. and Solanum species. In the most preferred embodiment, the Piper species is Piper betel. Similarly, the Solanum species is any one or combination of S. mammosum, S. verbascifolium, S. tuberosum, S. trilobatum, S. torvum, S. seaforthianum, S. sarmentosum, S. nigrum, S. melongena, S. maroniense, S. microcarpon, S. jasininoides, S. indicum, S. ferox, S. coagulans, S. blumei, S. aculeatissimum, while the tropical seaweed is any one or combination of Eucheuma cottonii, Culerpa lentillifera, Appaphycus alvareziii and Sargassum polycystum. The inventors of the present invention found that the alcohol soluble extract derived from the aforementioned species possesses both acceptable taste that confers the derived extract to be comfortably incorporated with the comestibles product, capsules, tablets or topical medicine with minimal additional refining process.

In one of the aspect, the Palm species is Elaeis guineensis, Elaeis oleifera, Phoenix dactylifera and Cocos nucifera. Moreover, it is the leaves of these species used for extracting the useful compounds.

According to the preferred embodiment, the extract to be incorporated into the comestibles and medicine can be acquired from any known method not limited only to the foregoing disclosed method. Following to another embodiment, the extract is prepared in a concentrated form, preferably paste or powdery form which enables the extract to be incorporated in various formulations of the comestibles, capsules, tablets or topical products.

In line with the preferred embodiment, the extract shall be the plant metabolites which are susceptible to an extraction solvent. The compounds and small peptides with the anti-cancer and cardiovascular system health-promoting properties are those metabolites with polarity in the alcoholic extracts. Therefore, the extract from the alcoholic extracts of plant parts of tropical seaweeds, Solanum spp. and Piper spp. and Palmae family is preferably derives from the extraction solvent of water, alcohol, acetone, chloroform, liquid CO₂ and any combination thereof.

In view of the prominent property of promoting anti-cancer and general healthcare of the cardiovascular system by the extracts in a subject, further embodiment of the present invention includes a method comprising the step of administrating orally or topically to the subject an effective amount of an extract derived from plant/s of tropical seaweeds, Solanum spp. fruit, palm spp. and Piper spp. Leaves.

The following example is intended to further illustrate the invention, without any intent for the invention to be limited to the specific embodiments described therein.

### Example 1

Vegetative parts of the plants were collected, cleaned, washed and cut into small pieces and oven dried at 40° C. overnight. The dried material was ground using a blender and extracted three times with hot and cold alcohol (1:10 v/v) and three times with hot and cold water or with mixtures of chloroform and alcohol. Other solvent such as acetone may be used as a medium for the extraction. This is a process designed to separate soluble compound by diffusion from a solid matrix (plant tissue) using a liquid matrix (solvent). Alcohol, water, chloroform and acetone has produced good yield in extracting the active components. The extraction was done a few times. The pooled extracts were vacuum-dried at 40° C. and stored until used.

### Example 2

The alcoholic extract of all these three tropical seaweeds species tested showed no toxicity to normal Vero cell line (Table 1). The alcoholic tropical seaweeds extracts exhibited dose and time depended inhibition against the proliferation of the five tested cancer cell lines. The IC50 values (Table 1) for breast cancer MDA-MB 231 cell line, were approximately 50 μg/ml for S. Polycystum, 60 μg/ml for C. lentillifera, and 100 μg/ml for E. Cottonii. The IC50 values for cervical cancer HeLa cell line, were approximately 30 μg/ml for S. Polycystum, 55 μg/ml for C. lentillifera, and 70 μg/ml for E. Cottonii. Amongst the tropical seaweeds extracts S. Polycystum extract (SPME) showed the strongest anti-proliferation activity against all the cancer cell lines tested with IC50 values of 25, 50, 30, 100, 150 μg/ml after 24 h treatment on breast cancer MCF-7, MDA-MB-231, HeLa, liver cancer HepG2, and colon cancer HT-29 cells, respectively. A very good correlationship were found between the cancer cells anti-proliferation activity with the extracts total polyphenols content, and electron donor activities but not with their proton donor activities.

TABLE 1
The IC50 values of tropical seaweeds alcoholic dry extracts on various
human cancer cell lines expressed as μg/ml, and Cytotoxicity on Vero cells

| | Incubation | IC50(μg/ml) | | |
|---|---|---|---|---|
| | Time | S. | C. | E. |
| Cell line | (hr) | polycystum | lentillifera | cottonii |
| MCF-7 | 24 | 25 ± 0.1 | 30 ± 0.6 | 47 ± 0.1 |
| | 48 | 22 ± 0.3 | 30 ± 0.9 | 40 ± 0.4 |
| | 72 | 20 ± 0.2 | 25 ± 0.5 | 30 ± 0.5 |
| HeLa | 24 | 30 ± 0.3 | 55 ± 0.1 | 70 ± 0.4 |
| | 48 | 25 ± 0.4 | 48 ± 0.1 | 60 ± 0.8 |
| | 72 | 20 ± 0.1 | 40 ± 0.7 | 50 ± 0.4 |
| MDA-MB- | 24 | 50 ± 0.4 | 60 ± 0.8 | 100 ± 0.3 |
| 231 | | | | |
| | 48 | 50 ± 0.6 | 50 ± 0.6 | 90 ± 0.2 |
| | 72 | 42 ± 0.3 | 40 ± 0.2 | 80 ± 0.4 |
| HepG2 | 24 | 100 ± 0.1 | 110 ± 0.6 | 100 ± 0.5 |
| | 48 | 95 ± 0.5 | 100 ± 0.4 | 90 ± 0.6 |
| | 72 | 90 ± 0.8 | 100 ± 0.6 | 90 ± 0.1 |
| HT-29 | 24 | 150 ± 0.2 | 200 ± 0.2 | >200 |
| | 48 | 150 ± 0.3 | 150 ± 0.3 | >200 |
| | 72 | 120 ± 0.5 | 120 ± 0.9 | >200 |

Maximum non-toxic concentration on Vero cells
(μg/ml) by observing morphological changes

| | | | |
|---|---|---|---|
| Vero cells | 1000 | 1250 | 1000 |

Values are expressed as Mean ± Standard deviation, n = 3.

The methanolic tropical seaweeds extracts exhibited dose and time dependent inhibition against the proliferation of the breast cancer cell lines with IC50 values of 47 μg/ml after 24 h treatment.

In a preliminary study we showed that the methanolic tropical seaweeds (Eucheuma cottonii) extracts have cytotoxic effect on the five human cancer cell lines (MCF-7, MDA-MB-231, HeLa, HepG2, and HT-29). The estrogen +ve human breast cancer MCF-7 cell line was the most sensitive and the human colon carcinoma HT-29 was the most resistant cell line to this tropical seaweeds extract. These results showed that the extracts of the tropical seaweeds selectively inhibited the growth of particular or tumour cells. These results suggested that the active substances from tropical seaweeds interact with special cancer-associated receptors or cancer cell specific molecules, to trigger the mechanisms leading to cancer cell death.

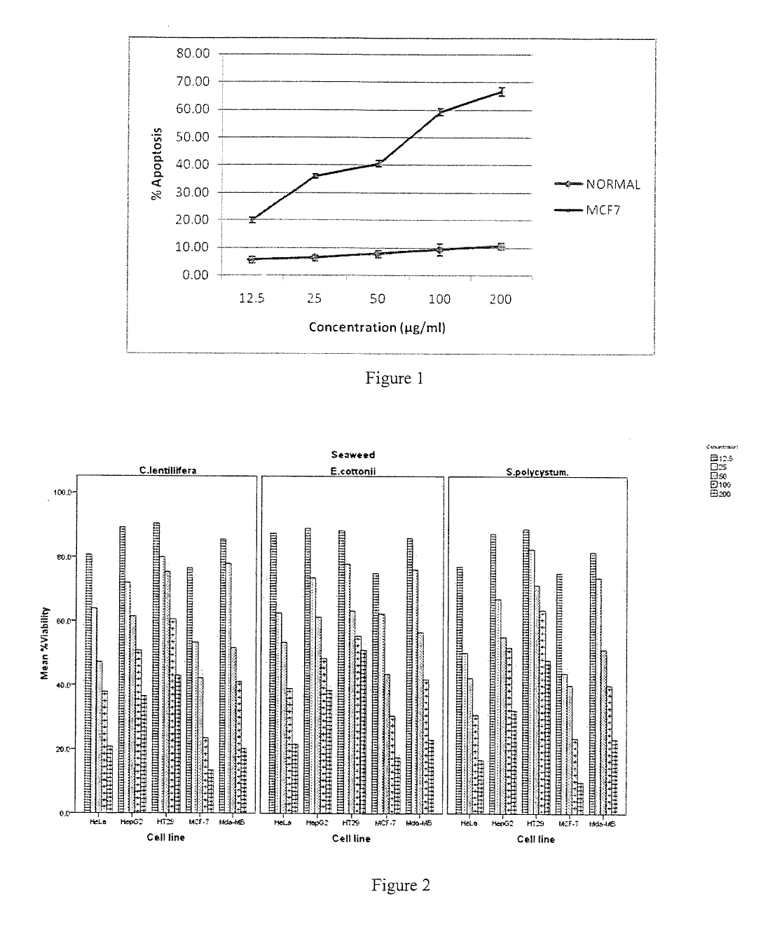

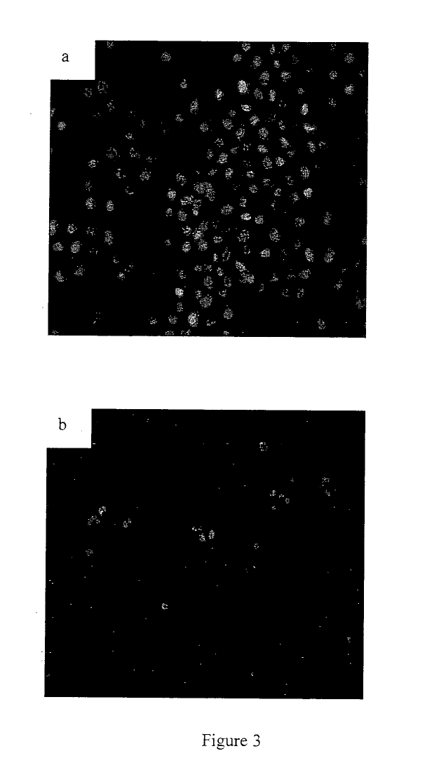

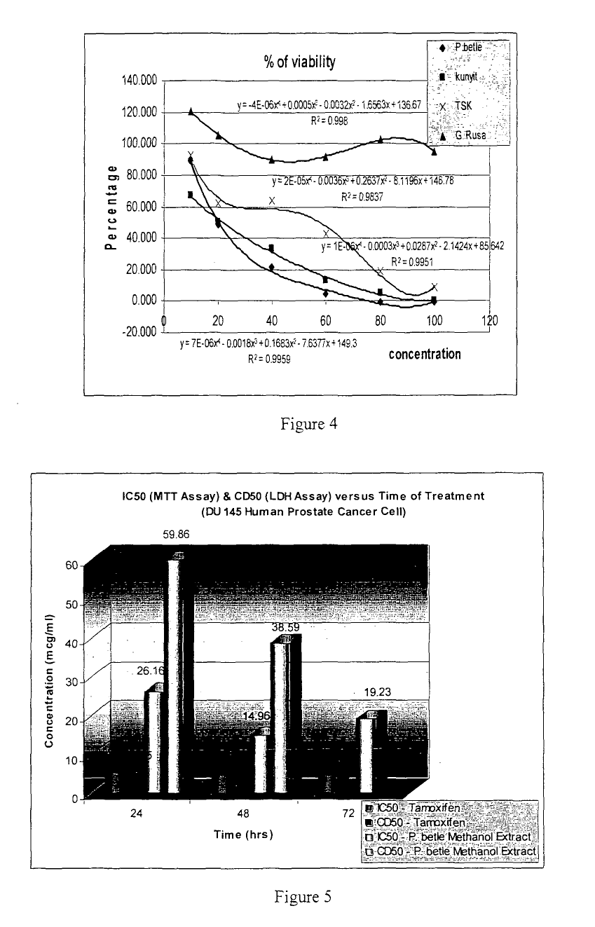

Percentages of apoptotic MCF-7 cells in the presence of ECME (0-200 μg/ml) compared to normal cells are depicted in FIG. 1. The percentages of apoptotic cells increased from 18% to 76% by increasing the concentration of the tropical seaweeds extract from 40 to 200 μg/ml.

### Example 3

Percentages of apoptotic MCF-7 cells in the presence of SPME (0-200 μg/ml) compared to normal cells are depicted in FIG. 1. Apoptosis (natural suicidal cell death) was evaluated using fluorescence microscopy by calculating the percentage of cells showing nuclear morphology of apoptosis after staining with Hoechst 33342. Percentages of apoptotic cells increased from 20% to 68% by increasing the concentration of the tropical seaweeds extract from 12.5 to 200 μg/ml. This increasing apoptosis did not occur in normal cell lines. The MTT assay showed them to be cytotoxic against all the cell lines in a dose dependent manner, with brown tropical seaweeds (S. polycystum) having the greatest inhibition. Cytotoxicity was not observed in normal Vero cell line. The estrogen positive breast cancer MCF-7 and human cervical adenocarcinoma HeLa cell lines were most sensitive to S. Polycystum crude methanol extract (SPME), with IC50 of 20 μg/ml, after 72 hours incubation. The IC₅₀ value of SPME on MCF-7, MDA-MB-231, HeLa, HepG2, and HT-29 cells was approximately 25, 50, 30, 100, 150 μg/ml after treatment for 24 h. Treatment of MCF-7 cells with various concentrations of SPME resulted in growth inhibition and induction of apoptosis in a time- and dose-dependent manner. The cancer cells anti-proliferative activities in the extracts were due to the phenolic compounds and electron donor free radical scavenging activities.

The cytotoxicity of the tropical seaweeds extracts was evaluated by examining the method of growth inhibition. When the growth inhibited cells were stained with Hoechst 33342, apoptotic cell death was observed in all the cancer cell cultures, and suggested that ECME caused irreversible damages in the cultured cancer cells.

### Example 4

Tropical seaweeds administration lengthened the rat estrous cycle from 4.2±0.82 to 5.8±1.1 at 150 mg/kg body wt. and to 5.6±1.8 at 300 mg/kg body wt. The Institutional Animal Care and Use Committee (IAUC) of Faculty of Veterinary Medicine at University Putra Malaysia (UPM) approved all the experimental procedures, and all animal research were conducted according to the Guidelines for the Care and Use of Laboratory Animals. Rats were allowed for acclimatization a week before the induction. Previous reports suggest that exposure to estrogens and an imbalance in the estrogen/progesterone ratio may be the most critical determinants in risk of estrogen-dependent diseases. Japanese women (who have one of the lowest rates of breast, endometrial, and ovarian cancers in the world) have longer menstrual cycles and lower serum estradiol levels than their Western women. To date, these low rates have been partly attributed to the soy-rich diets inherent among Asian populations. Previous studies investigating the role of dietary soy or genistein on the rat estrous cycle showed either no effects or only a modest 10% increase in cycle length, suggesting that tropical seaweeds may exert a greater effect in increasing cycle length than soy intake. The tumour (cancer) incidence rate of each group respectively was 87.5% (⅞) in control untreated group, 37.5 (⅜) in low 150 mg/kg dose Euchema cottonii methanolic extract (ECME) group, 12.5% (⅛) in high 300 mg/kg dose ECME group. Statistical analysis showed that the tumor incidence rates of both treated groups were significantly lower than that of control group. The total tumor volume of each group respectively was: 9.8 cm3 in control untreated group, 0.95 cm3 in 300 mg/kg ECME group, 2.4 cm3 in 150 mg/kg ECME group. Statistical analysis showed that the total tumor volume of all treated groups were significantly (P<0.05) smaller than that of control untreated group.

### Example 5

An experiment whereby 40 rats were randomly divided into 5 groups consist of eight rats in each group, with one negative control group (normal diet) was conducted (FIG. 1). Rat mammary gland tumour were induced using rat mammary gland cancer cells inoculated subcutaneously into mammary fat pad (right flank) of female Sprague-Dawley rats with a 200 μl of cell mixture (total 6×10⁷ cells) using a 26-gavage needle. Oil palm fronds extract (OPFE) were dissolved in water and administered orally. All animals were inspected daily, while body weights were recorded weekly. Rats were palpated weekly to monitor tumor development. The diameters of each tumor were measured with calipers and tumor volume was calculated using the following formula: largest diameter×(smallest diameter)²×0.4.

TABLE 2
Experimental procedures and mammary tumors among the groups at autopsy

| | | | Pre- | | Post- | | |
|---|---|---|---|---|---|---|---|
| | | | inoculation | | inoculation | | Total |
| | | Acclimation | Treatment | Inoc- | Treatment | Incidence | Volume |
| Group Name | Group Details | (1 week) | (4 week) | ulation | (2 week) | (%) | (cm3) |
| A = Crl | Control | + | − | + | − | 87.5% ^{a} | 10.7 |
| B = LPIT | Pre & post | + | + | + | + | 25% ^{c} | 1.4 |
| | inoculation | | | | | | |
| | with low | | | | | | |
| | dietary OPFE | | | | | | |
| | (150 mg/kg) | | | | | | |
| C = H.PIT | Pre & Post | + | + | + | + | 12.5% ^{d} | 0.8 |
| | inoculation | | | | | | |
| | with high | | | | | | |
| | dietary OPFE | | | | | | |
| | (300 mg/kg) | | | | | | |
| D = L,PoIT | Post-Inoculation | + | − | + | + | 37.5% ^{b} | 2.6 |
| | with low | | | | | | |
| | dietary OPFE | | | | | | |
| | (150 mg/kg) | | | | | | |
| E = H.PoIT | Post-Inoculation | + | − | + | + | 25% ^{c} | 0.9 |
| | with high | | | | | | |
| | dietary OPFE | | | | | | |
| | (300 mg/kg) | | | | | | |

Incidence rate followed by different superscript alphabets were significantly different at 5% level.

The tumour incidence rate of each group respectively was 87.5% (⅞) in control group, 25% ( 2/8) in low dose 150 mg/kg pre and post inoculation treatment (L-PIT) group, 12.5% (⅛) in high dose 300 mg/kg pre and post inoculation treatment (H-PIT) group, 37.5% (⅜) in low dose 150 mg/kg post inoculation treatment (L-PoIT) group, and 25% ( 2/8) in high dose 300 mg/kg post inoculation treatment (H-PoIT) group. Statistical analysis showed that the tumor incidence rates of H-PIT, L-PIT, H-PoIT and L-PoIT groups were significantly (P<0.05) lower than that of control group. The tumor incidence rate of the L-PoIT group was significantly higher than the other treated groups. The difference of the tumor incidence rate of L-PIT group and H-PoIT group did not have difference significance.

The total tumor volume of each group respectively was: 10.7 cm3 in control group, 0.8 cm3 in H-PIT group, 1.4 cm3 mL-PIT group, 0.9 cm3 in H-PoIT group and 2.6 cm3 in L-PoIT group. Statistical analysis showed that the total tumor volume of all treated groups were significantly smaller than that of the untreated control group. The total tumor volume of L-PoIT group and L-PIT group were significantly bigger than that of high dose groups. POFE showed an anti-proliferation effect on the growth of MCF-7 cells at medium and high concentration like estrogen. The results showed that consumption of POFE benefitted the animals in preventing new development of breast cancer or therapeutic treatment of a pre-existing one. The benefits of consumption of the plant product thus indicate a possible component in a prevention and treatment strategy. In the present study, we found the tumor incidence, and the sums of tumor volume in all treatments groups were lower than that of control group. We previous showed, oil palm frond methanolic extract contained high phenolic and flavonoid compounds and moderate antioxidant activities.

### Example 6

Six selected tropical edible plants (Anacardium occidentale, Morinda eliptica, Cymbopogon citratus, Piper betle, Carica papaya and Elaeis guineensis) were compared for anti-cancer activity using the MTT assay (3-[4,5-dimethylthiazol-2-yl]-2,5-di-phenyl tetrazolium bromid). The MTT assay is used to determine the cytotoxic property of methanol plant extracts on “MDA-MB-435 estrogen receptor-negative human breast cancer” cells using the IC₅₀ (50% Inhibitory Concentration) value as an indicator of their effectiveness. The strongest anti-cancer effects were shown by the Piper betle leaf extract that exhibited IC₅₀ value of 18 (±9.07) μg/ml against the MDA-MB-435 estrogen receptor-negative human breast cancer cell, followed by Carica papaya [66.00 (±8.48) μg/ml], Morinda eliptica [68.25 (±9.18) μg/ml], Cymbopogon citratus [71.00 (±9.15) μg/ml], and Elaeis guineensis [75.00 (±8.63) μg/ml]. Anacardium occidentale showed no measurable inhibition by MTT assay. All the edible tropical plant leaves showed no cytotoxicity on normal human cell lines.

1-13. (canceled)

14. A composition with anti-cancer properties comprising an alcohol soluble extract from leaves selected from the group consisting of Piper species, Palm species, and combinations thereof.

15. A composition according to claim 14, further comprising an alcohol soluble extract from plant parts of Solanum species and/or tropical seaweeds.

16. A composition according to claim 15, wherein the Solanum species is selected from the group consisting of S. mammosum, S. verbascifolium, S. tuberosum, S. trilobatum, S. torvum, S. seaforthianum, S. sarmentosum, S. nigrum, S. melongena, S. maroniense, S. microcarpon, S. jasminoides, S. indicum, S. ferox, S. coagulans, S. blumei, S. aculeatissimum, and combinations thereof.

17. A composition according to claim 15, wherein the tropical seaweeds is selected from the group consisting of Kappaphycus species, Euchema species, Caulerpa species, Sargassum species, and combinations thereof.

18. A composition according to claim 14, wherein the Palm species is selected from the group consisting of Elaeis species, Phoenix species, Cocos species, and combinations thereof.

19. A method for obtaining an alcohol soluble extract with anti-cancer properties from plant parts of Piper species, palm species, Solanum species and/or tropical seaweed, comprising:
pre-treating plant parts of the Piper species, Solanum species and/or tropical seaweed;
subjecting the pre-treated plant parts for extraction using a first polar extraction solvent comprising an alcohol;
subject the pre-treated plant parts using a second polar extraction solvent comprising water; and
concentrating the first and second polar extraction solvent to form a powdery or paste-like composition after removing the pre-treated plant parts.

20. A method according to claim 19, wherein the Solanum species is selected from the group consisting of S. mammosum, S. verbascifolium, S. tuberosum, S. trilobatum, S. torvum, S. seaforthianum, S. sarmentosum, S. nigrum, S. melongena, S. maroniense, S. microcarpon, S. jasminoides, S. indicum, S. ferox, S. coagulans, S. blumei, S. aculeatissimum, and combinations thereof.

21. A method according to claim 19, wherein the Palm species is selected from the group consisting of Elaeis guineensis, Elaeis oleifera, Phoenix dactylifera, Cocos nucifera, and combinations thereof.

22. A method according to claim 19, wherein ratio of weight of the pre-treated plant parts to the first polar extraction solvent is in a range of 1-3:6-10 (w/v).

23. A method according to claim 19, wherein the plant parts of the Piper species are leaves.

24. A method according to claim 19, wherein the tropical seaweeds is selected from the group consisting of Kappaphycus species, Euchema species, Caulerpa species, Sargassum polycystum, and combinations thereof.
